SEQUENCE LISTING (22 sequences)

SEQ ID NO: 1 - DNA, 20 bp, Artificial Sequence
  Primer
atttgttacataatctggtt

SEQ ID NO: 2 - DNA, 20 bp, Artificial Sequence
  Primer
cctgggtcttataaagtcct

SEQ ID NO: 3 - DNA, 21 bp, Artificial Sequence
  Primer
ccagcaccgtcttcccaggtt

SEQ ID NO: 4 - DNA, 22 bp, Artificial Sequence
  Primer
catagccgaagccatagcccac

SEQ ID NO: 5 - DNA, 23 bp, Artificial Sequence
  Primer
ccatcactgccacccagaagact

SEQ ID NO: 6 - DNA, 23 bp, Artificial Sequence
  Primer
gcaggtcagatccacaacggaca

SEQ ID NO: 7 - DNA, 27 bp, Artificial Sequence
  Primer
gccgttgtagccatagccgaagccata

SEQ ID NO: 8 - DNA, 28 bp, Artificial Sequence
  Primer
gtactaccgtagccacagcccacactgt

SEQ ID NO: 9 - DNA, 28 bp, Artificial Sequence
  Primer
gtcttcccaggttgctactggggtagct

SEQ ID NO: 10 - DNA, 28 bp, Artificial Sequence
  Primer
gtgggctgtggctacggtagtacctact

SEQ ID NO: 11 - DNA, 20 bp, Artificial Sequence
  Primer
cctttaggattgactgattt

SEQ ID NO: 12 - DNA, 20 bp, Artificial Sequence
  Primer
tttcactctctctttcacaa

SEQ ID NO: 13 - DNA, 20 bp, Artificial Sequence
  Primer
aagagagagtgaaaatcgct

SEQ ID NO: 14 - DNA, 20 bp, Artificial Sequence
  Primer
atgaatgggaacttttacct

SEQ ID NO: 15 - DNA, 20 bp, Artificial Sequence
  Primer
atttgttacataatctggtt

SEQ ID NO: 16 - DNA, 20 bp, Artificial Sequence
  Primer
cctgggtcttataaagtcct

SEQ ID NO: 17 - DNA, 20 bp, Artificial Sequence
  Primer
aacaacccatcctagtattc

SEQ ID NO: 18 - DNA, 20 bp, Artificial Sequence
  Primer
ggagtaggtactaccgtagc

SEQ ID NO: 19 - DNA, 559 bp, Ovis aries
atactgaggaaattcattccctgctctccaagccgcccaacccagacaccatgagctactgcttctccagcaccgtcttcccaggttgctactggggcagctatggctacccgctgggctacagtgtgggctgtggctacggtagtacctactccccagtgggctatggcttcggctatggctacaacggctctggggccttcggttgccgaagattctggccatttgctctctactgatttgctgaaataccagaggcatggaatcttctcccccaaacccacgaggcggacttccaggtcctcagagactcatcagcctcccagttagctgcttttcacatcggcaccagagtctcaagggaagaagatgaaaaaccacttgcctccagctgccttccctgcatgatgtttgttgggacattttgagaaacttgacacccaaacacgttttacgtttgaatttttcaccatgctcatgactcttgtcattatcaagttgtggatgtgtctgtcaaattctcaataaacttgtctcaaccgcaaaaaaaaaaaaaaaa

SEQ ID NO: 20 - DNA, 54 bp, Ovis aries
tgagcacgcgcacacacacacacacacacacacacacacacacagtatgtatat

SEQ ID NO: 21 - DNA, 67 bp, Ovis aries
cgcgcgcacgcacacacacacacacacacacacacacacacacacacacacacacacaagtatgtat

SEQ ID NO: 22 - DNA, 69 bp, Ovis aries
tgagcacgcgcacacacacacacacacacacacacatacacacacacacacacacacacagtatgtatt

(19) United States
(12) Patent Application Publication
(10) Pub. No.: US 2021/0332443 A1
(43) Pub. Date: Oct. 28, 2021

(54) Molecular Marker and Application Method for Assisted Breeding of Fine-wool Sheep

(71) Applicant: China Jiliang University, Hangzhou (CN)

(72) Inventors: Feng Guan, Hangzhou (CN); Xinyu Hu, Hangzhou (CN); Nan Wang, Hangzhou (CN); Yuting Jin, Hangzhou (CN); Guoqing Shi, Hangzhou (CN); Pengcheng Wan, Hangzhou (CN); Rong Dai, Hangzhou (CN); Aichun Xu, Hangzhou (CN); Jian Ge, Hangzhou (CN); Jun Liu, Hangzhou (CN)

(73) Assignee: China Jiliang University, Hangzhou (CN)

(21) Appl. No.: 17/009,867

(22) Filed: Sep. 2, 2020

(30) Foreign Application Priority Data
Apr. 28, 2020 (CN) 2020103483112

(51) Int. Cl. C12Q 1/6888

(52) U.S. Cl. CPC C12Q 1/6888; C12Q 2600/124

(57) ABSTRACT

A molecular marker for the selection and breeding of fine-wool sheep is disclosed. The marker is an STR marker comprising a (CA)ₙ repeat core sequence, with n between 5 and 24 that can be obtained by PCR amplification of genomic DNA of sheep using the primers shown in SEQ ID NO: 1 and SEQ ID NO: 2 and sequencing the PCR product. When n is 17 or 18, a sheep is a fine-wool breed, and when n is 23 or 24, a sheep is a non-fine-wool breed. When the CA repeat is discontinuous, i.e., divided into two segments (e.g., 12+11 or 13+11) separated by two bases TA or GA, a sheep is a hybrid breed of fine-wool sheep and non-fine-wool sheep. Use of the marker provides methods of identifying of fine-wool sheep breeds, and efficient and accurate selection of fine-wool sheep or the hybrid offspring of fine-wool sheep for breeding.

### SEQUENCE LISTING

Incorporated by reference herein in its entirety is a computer-readable sequence listing submitted via EFS-Web and identified as follows: One (4,949 byte ASCII (Text)) file named “2020_09_10_sequence_listing.txt” created on Sep. 10, 2020.

### FIELD OF THE INVENTION

This invention relates to molecular marker-assisted breeding technology for livestock, specifically, a fine-wool sheep screening and application method using trait function related gene, i.e., Keratin associated protein 8 (KAP8) gene and its related STR locus.

### BACKGROUND

As a natural fiber, wool plays an important role in animal husbandry and textile industry and influences the direction of sheep breeding to a great extent. All the time, the breeding of fine-wool sheep mainly depends on the phenotype and progeny testing, therefore, there is slow progress in selection intensity and breeding. With the development of biotechnology and the extensive research on genomics and proteomics of wool, the analysis for the wool composition, encoding gene and expression regulation has been made for many sheep breeds in the world, and the major progress has been made in the composition and structure of wool, genetic structure and developmental regulation process by combining with the research achievements made for the hair component and structure of other animals. These results have provided the foundation and possibility for the molecular breeding of fine-wool sheep.

In terms of structure, wool is generally divided into wool epidermis (i.e. scale layer), hair cortex (i.e. cuticle) and medulla layer from outside to inside, and generally, fine wool does not have medulla. Although most wool have a three-layer structure, the wool fiber entity is mainly composed of cuticle, which accounts for 90% or even more than 98% of the weight of clean wool fiber. A microscopic analysis further shows that the cells of cuticle can be divided into three types—orthocortex, paracortex and mesocortex, all of which are composed of keratin. According to the structure and composition of wool protein, it is the α-helical intermediate filament proteins (IFPs) that constitute the skeleton structure of wool fiber and the keratin associated proteins (KAPs) that serve as the filling component of matrix. The α-helical IFPs accounts for more than 50% of the total amount of wool protein with stable content, while the KAPs connects the α-helical IFPs transversely into microfilament bundle through disulfide bond. Both proteins in cross link finally forms wool fiber. In terms of the difference in the composition of wool with different fineness and curvature, it was found that the high glycine-tyrosine proteins (HGTPs) were one of the main differential proteins, which belong to KAP family. The HGTP subfamily mainly includes KAP6, KAP7 and KAP8 which are the important target genes for studying the growth and development of wool and the molecular breeding of fine-wool sheep because their gene expression levels directly affect the fineness and curvature of wool, while KAP8 is recognized as the main regulatory gene for the wool development of cashmere goats. Therefore, it becomes an important research direction for molecular-assisted breeding of fine-wool sheep to screen KAP6, KAP7, and KAP8 genes, in particular the regulatory sequences or markers directly related to KAP8.

Microsatellite locus, also known as short tandem repeats (STRs) or simple sequence repeats (SSR), whose basic constitution unit is 2 to 6 bp, and 2 to 4 nucleotide repeat sequence are more common, is widely distributed in eukaryotic genomes and mostly located near the coding region. With many advantages such as high interspecific conservation, co-dominant inheritance, high polymorphism and allele complying with Mendel's law of inheritance, STR markers are widely used in the research, such as mapping of animal genes and linkage analysis of excellent traits, and multiple STR loci have been applied for the assisted breeding of excellent traits of livestock, which has greatly improved the breeding efficiency.

### SUMMARY OF THE INVENTION

The purpose of the invention is to provide a molecular marker and application method for assisted breeding of fine-wool sheep, so as to use the method for the early screening and molecular-assisted breeding of fine-wool sheep individuals.

The invention firstly provides a STR molecular marker for assisted breeding of fine-wool sheep. The mentioned STR marker is a CA base repeat in the upstream region of sheep KAP8 gene, which has obvious species specificity because its repeat number differs between the fine-wool sheep and other sheep breeds;

In terms of STR marker mentioned in the invention, the number of repeat of CA base repeat is between 5 and 24; the mentioned STR marker is located in the upstream region of sheep KAP8 gene, and the last CA repeat is 458 bp away from the transcription starting point of KAP8 gene;

The STR marker provided by the invention is used as a DNA molecular marker for the breeding of fine-wool sheep, which can be used for the identification of fine-wool sheep breed, non-fine-wool sheep breed and the hybrid offspring;

On the other hand, the invention provided the primer pair for detecting the above-mentioned STR marker;

Where, a kind of specific sequence information of the primer pair is as follows:

(SEQ ID NO. 1)
5′-ATTTGTTACATAATCTGGTT-3′
(SEQ ID NO. 2)
5′-CCTGGGTCTTATAAAGTCCT-3′

The invention also provides a breeding method for fine-wool sheep, which is to implement the breeding by detecting the above-mentioned STR molecular marker through primer set amplification. When the number of repeat (CA) in the PCR amplification product is 17 or 18, the sheep to be tested is fine-wool sheep, and when the number of repeat (CA) is 23 or 24, the sheep to be tested is non-fine-wool sheep; when the number of repeat (CA) is in the range 5≤n≤24 and the CA repeat sequence is discontinuous repeat, the sheep to be tested is the hybrid offspring of fine-wool sheep and non-fine-wool sheep.

The specific steps of one of the above mentioned method is as follows:

1) Obtain genomic DNA from the sample to be tested;

2) Carry out PCR amplification for the DNA obtained in step 1) using the primers SEQ ID NO.1 and SEQ ID NO.2 which are used to test STR marker;

3) Carry out DNA sequencing for the PCR amplification product obtained in step 2). When n, the number of repeat (CA) in the PCR amplification product is 17 or 18, the sheep to be tested is fine-wool sheep, and when n is 23 or 24, the sheep to be tested is non-fine-wool sheep; when n, the number of CA repeat sequence is in the range 5≤n≤24 and the CA repeat sequence is discontinuous repeat, the sheep to be tested is the hybrid offspring of fine-wool sheep and non-fine-wool sheep.

Where, the system of PCR amplification system in step 2) is as follows:

The total volume is set to 20 μL, including 2.0 μL Buffer solution (10×, including 20 mM MgCl₂), 0.1 μL˜3 μL specific primer pair respectively (SEQ ID NO. 1 and SEQ ID NO. 2, with the concentration of 20 μM), 0.2 μL˜4 μL dNTP mix (with the concentration of 2.5 mM), 1.0 μL˜5 μL genome DNA (about 40 ng DNA), 0.2 μL˜1 μL Taq DNA polymerase (5 U/μL) and adding ddH₂O until to 20 μL;

PCR amplification conditions are:

Denaturation for 5 min at 95° C.; denaturation for 30 s at 95° C., annealing for 30 s˜40 s at 47° C.˜55° C. and extension 30 s˜40 s at 72° C., which serve as a cycle, with implementing 28˜35 cycles; then extension 5 min at 72° C.; and then cool down at 4° C. to save for later use.

For further improvement of the invention, a molecular marker and application method for assisted breeding of fine-wool sheep:

The PCR amplification system is:

The total volume is set to 20 μL, including 2.0 μL Buffer solution (10×, including 20 mM MgCl₂), 0.1 μL specific primer pair respectively (SEQ ID NO. 1 and SEQ ID NO. 2, with the concentration of 20 μM), 1.2 μL dNTP mix (2.5 mM), 0.2 μL Taq DNA polymerase (5 U/μL), 2 μL DNA template (including about 40 ng DNA) and adding water until to 20 μL;

PCR amplification conditions are:

Denaturation for 5 min at 95° C.; denaturation for 30 s at 95° C., annealing for 30 s at 52.5° C. and extension 35 s at 72° C., which serve as a cycle, with implementing 30 cycles; then extension 5 min at 72° C.; and then cool down at 4° C.

Another improvement for the invention, a molecular marker and application method for assisted breeding of fine-wool sheep:

A negative control is set up in the mentioned step 2), which should be the double distilled water reaction system without DNA from any source.

The invention obtains through screening the associated STR locus of KAP8 gene directly related to wool development, which is located in the upstream of coding region of KAP8 gene. Through the association with the difference of species specificity, it can be used for the identification of the fine-wool sheep breed and the assisted selection of crossbreeding to achieve the breed identification and early breeding, so as to reduce the breeding cost and accelerate the breeding process. The method in the invention is easy and with high accuracy and low cost, and greatly shortens the testing time than that of progeny testing.

### BRIEF DESCRIPTION OF THE DRAWINGS

FIG. 1 is the Standard Curve Diagram of Relative Quantification of KAP8 Gene Expression of Sheep in Implementation Example 2,

FIG. 2 is the Melting Point Curve (Tm=84° C.) Diagram of GAPDH Primer Amplification of Reference Genes in Implementation Example 2,

FIG. 3 is the Melting Point Curve (Tm=87° C). Diagram of Primer Amplification of KAP8 Gene of Sheep in Implementation Example 2,

FIG. 4 is the Structure Diagram of KAP8 Gene (SEQ ID NO: 19) of Sheep in Implementation Example 3,

FIG. 5 is the Figure of Amplification Product of KAP8 Gfl-KAP8 Grl Primer Pair in Implementation Example 5, where M represents the molecular Marker, and lanes 1-3 respectively are Chinese Merino fine wool sheep, Suffolk sheep and goat.

FIG. 6 is the Result Diagram of (CA) 17 sequencing of Chinese Merino fine wool sheep in Implementation Example 5, (SEQ ID NO: 20)

FIG. 7 is the Result Diagram of (CA) 24 sequencing of Suffolk sheep in Implementation Example 5, (SEQ ID NO: 21)

FIG. 8 is the Result Diagram of (CA) 13+11 Sequencing of Meat-prolificacy Strain of Chinese Merino fine wool sheep Crossed with Hu Sheep in Implementation Example 5 (SEQ ID NO: 22).

### DETAILED DESCRIPTION

The contents included in the invention are as follows:

(1) Collect the fine-wool sheep and control samples. Chinese Merino fine wool sheep (reclamation type) was selected as the representative of the fine-wool sheep, and Hu sheep, Suffolk sheep and the hybrid offspring of Chinese Merino fine wool sheep were selected as the representatives of the control samples, and also the goat DNA samples. The ear tissue and the skin tissue of left shoulder of each individual was taken respectively, and then extracted genome DNA and total RNA for DNA and RNA analysis.

(2) Comparison of the KAP8 gene expression levels in the hair follicle tissue of Chinese Merino fine wool sheep and Suffolk sheep, so as to learn about the difference of KAP8 gene expression in two breeds.

(3) In order to analyze the structure of KAP8 gene, the full-length cDNA of KAP8 was cloned from Chinese Merino fine wool sheep through 5′-RACE and 3′-RACE technology.

(4) In order to analyze the difference in upstream regulatory sequences of KAP8 gene of Chinese Merino fine wool sheep, Suffolk sheep and other sheep breeds, about 2,500 bp upstream sequence of KAP8 gene of multiple sheep breeds and the hybrid offspring were amplified and the genetic polymorphism was analyzed, and the STR marker in the invention was found.

The method process of the invention will be further described below in combination with examples, but the examples are limited to illustrations and are not limited to the operations in the examples. The specific conditions and the experimental methods that have not been noted in the following implementation process usually can be carried out under conventional conditions such as the experimental conditions described in Molecular Cloning: A Laboratory Manual or according to the conditions suggested by the manufacturer. Relevant technicians in the field can better understand and master the invention by virtue of the examples. However, the scope of protection and claims of the invention are not limited to the examples provided below.

### IMPLEMENTATION EXAMPLE 1

### Collection and Processing of Sheep Samples

The sheep samples used for the invention include Chinese Merino fine wool sheep, Suffolk sheep, Hu Sheep, Chinese Merino meat-prolificacy strain, hybrid of Chinese Merino crossed with Hu Sheep. A total of 30 skin samples of ear tissue for each breed of sheep were collected and taken back to the laboratory in low-temperature environment (ice bag), and then extracted DNA using the animal tissue DNA extraction kit (Takara product) according to the operation instructions.

All extracted DNA were dissolved in the TE buffer in the kit, which was tested by Nanodrop 2000, and the result showed that the DNA concentration was varied 20˜400 ng/μL. Then the sample with the purity A₂₆₀/A₂₈₀=1.8˜2.0 was diluted to 20 ng/μL and stored in the refrigerator at 4° C. for later use. Otherwise, it should re-extract DNA until the concentration and purity met the needs of PCR amplification.

In the invention, the goat DNA was used as the control. The goat breed is Xuhuai goat, and the goat DNA should be the retained sample of the laboratory.

### IMPLEMENTATION EXAMPLE 2

### Expression Difference of KAP8 Gene in Chinese Merino Fine Wool Sheep and Suffolk Sheep

In order to test the influence of KAP8 on wool development and characteristics, three samples for each of Chinese Merino fine wool sheep and Suffolk sheep were selected to represent fine-wool sheep and coarse-wool sheep. Then shear the wool on left shoulder and disinfect with iodine tincture, and scissor skin tissue and separate the hair follicles, and then put the hair follicles into liquid nitrogen immediately for storage and later use. Extracted total RNA of sheep skin tissue by Trizol reagent was put into the refrigerator at −70° C. for storage and later use after the quality determination.

Design fluorescent quantitative PCR primer of KAP8 gene by referring to GenBank sequence X05639 of sheep KAP8:

Upstream Primer F:
SEQ ID NO. 3
5′-CCAGCACCGTCTTCCCAGGTT-3′:;
Downstream Primer R:
SEQ ID NO. 4
5′-CATAGCCGAAGCCATAGCCCAC-3″:;

The length of amplification product is expected to be 114 bp, and the reference primer is designed referring to GAPDH gene, whose sequence is as follows:

F:
SEQ ID NO. 5
5′ CCATCACTGCCACCCAGAAGACT-3:;
R:
5′ GCAGGTCAGATCCACAACGGACA-3′:

SEQ ID NO. 6; and the length of amplification product is expected to be 203 bp.

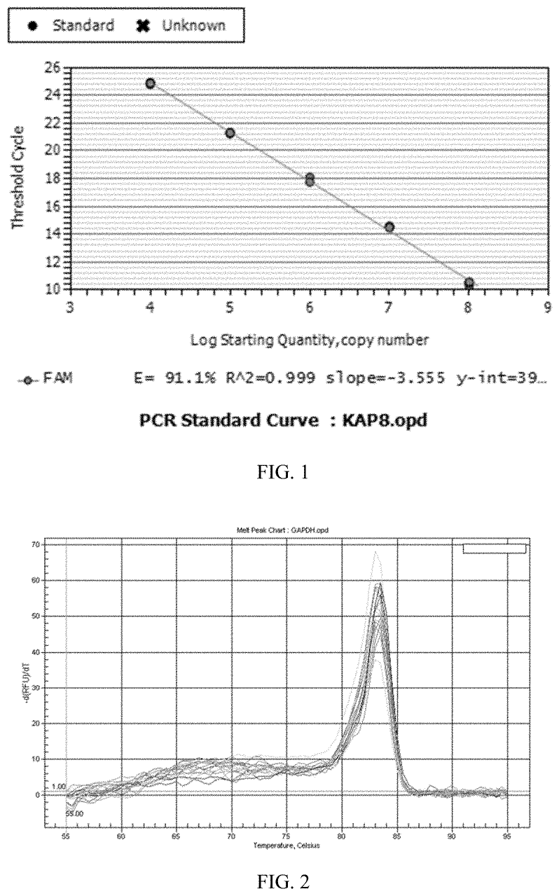

The cDNA of sheep skin tissue was synthesized by M-MLV Reverse Transcriptase cDNA synthesis kit (Promega) according to the instruction of kit. Carry out gradient dilution on the cDNA template, with diluting ten times for each gradient and five gradients in total. The optimized system is applied to implement RQ-PCR to obtain the standard curve of relative quantitation of KAP8 (FIG. 1) with Ct value as the ordinate and the log method of cDNA template number as the abscissa. Repeat the above procedures for three times for each sample, and the negative control was set up as well. The relative expression level of KAP8 gene was calculated through 2^{(Ct reference gene-Ct target gene)}, and the statistical analysis for data was made by the software SPSS 13.0.

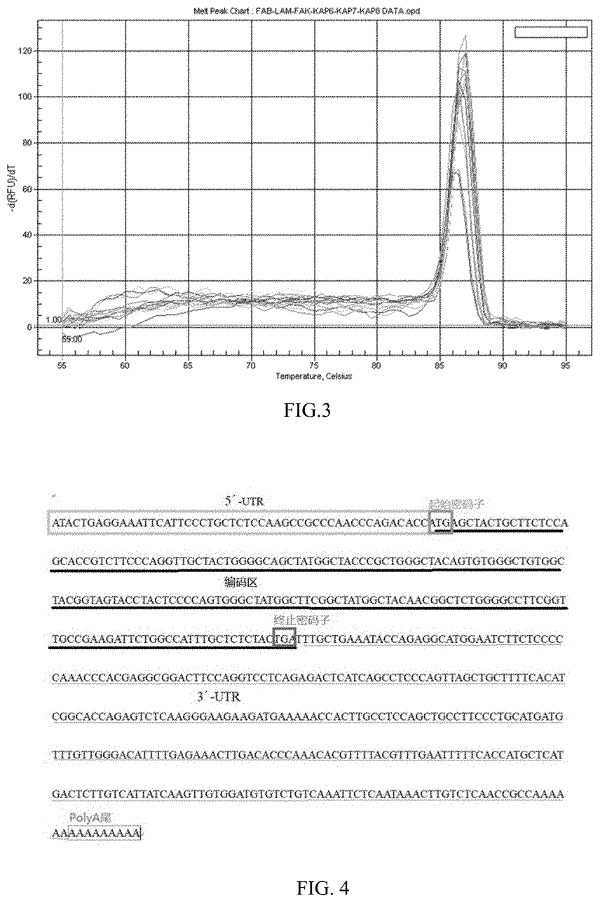

The result shows that the RQ-PCR primers of the reference gene and the target gene have high specificity, and there is no non-specific amplification (FIG. 2 and FIG. 3). The mRNA expression quantity of KAP8 gene is positively correlated with the wool diameter, and the correlation coefficient is 0.948 (P=0.00). The expression quantity in Suffolk sheep skin tissue is significantly higher than that in Chinese Merino fine wool sheep skin tissue (P<0.01), reaching 5.87 times, which indicated the expression levels significantly affects the fineness of wool. Therefore, further research on the regulation of KAP8 gene expression is needed to explore the molecular mechanism causing the expression difference, especially for the upstream regulatory sequence as the promoter region.

### IMPLEMENTATION EXAMPLE 3

### Full-Length cDNA Cloning and Analysis of Transcription Starting Point of KAP8 Gene

In order to make further research on the regulatory sequence of KAP8 gene and confirm the transcription starting point, the full-length cDNA of KAP8 gene was cloned through 5′-RACE and 3′-RACE technology.

In this experiment, firstly, total mRNA from Chinese Merino fine wool sheep obtained in Implementation Example 2 are reversely transcribed into cDNA by referring to the primers and instructions of the RACE kit (GeneRacer™ Kit (Invitrogen). The cDNA cloning for intermediate segment of KAP8 gene refers to the sequence No. X05639 in Genbank. The primer is designed by the software Primer Premier 5.0 and is synthesized by Hangzhou Qingke Biotechnology Co., Ltd. The primer sequence is shown in Table 1 below:

TABLE 1
Primer sequences for Full-length cDNA Clone of
KAP8 Gene

| | Name of | | SEQ |
|---|---|---|---|
| RACE | Primer | Sequence (5′-3′) | ID NO: |
| 5′- | rKAP8- | GCCGTTGTAGCCATAGCCGAAGCCATA | 7 |
| RACE | R1 | | |
| | rKAP8- | GTACTACCGTAGCCACAGCCCACACTGT | 8 |
| | R2 | | |
| 3′- | rKAP8- | GTCTTCCCAGGTTGCTACTGGGGTAGCT | 9 |
| RACE | F1 | | |
| | rKAP8- | GTGGGCTGTGGCTACGGTAGTACCTACT | 10 |
| | F2 | | |

According to the instructions of Kit, a nest-PCR was used in the 5′RACE of KAP8 gene. In the first round of this procedure, the PCR system included the synthesized and diluted 5′RACE cDNA as the template, and the primer pair was upstream 5′RACE outer primer supplied with the kit and the downstream was designed primer rKAP8-R1. In the second round of PCR, the DNA template was diluted PCR product in the first round, and the primer pair was 5′RACE inner primer used as upstream primer, its corresponding rKAP8-R2 primer was used as the paired primer. The 3′-RACE clone of KAP8 gene segment is also finished through nest-PCR according to the instruction, with the process basically similar with that of 5′RACE. PCR amplification products were sequenced by vector clone after separating on the 1.5% agarose gel.

The sequencing result showed that the fragment sizes of 3′RACE PCR product was 419 bp, and the fragment size of 5′RACE PCR product was 148 bp. The length of full-length cDNA of KAP8 gene was 559 bp, of which the length of 5′UTR and 3′UTR was respectively 50 bp and 305 bp and the length of CDS region was 189 bp. The CDS region includes the initiator codon ATG and the terminator codon TGA, with encoding 62 amino acids. The transcription starting point is located 50 bp in upstream of coding region, with basic A as the starting point. The full length of Sheep KAP8 gene was was 559 bp obtained by 5′RACE and 3′RACE clone technology, whose structure was shown in FIG. 4.

### IMPLEMENTATION EXAMPLE 4

### Cloning of the Upstream Region of KAP8 Gene and Discovery of STR Loci

After the transcription starting point in Implementation Example 3 is determined, in order to further analyze the upstream promoter sequence of the transcription starting point and its possible polymorphism difference, the sheep KAP8 gene (sequence No. X05639) in Genbank is used as the reference sequence, and then the Genome Walking kit (TaKaRa) is used and the thermal asymmetric interlaced PCR (TAIL-PCR) is applied to obtain the upstream sequence of coding region. Taking the obtained sequence as the DNA template, the primers (as shown in Table 2) were designed according to the transcription starting point determined in Implementation Example 3, and PCR amplifications were carried out for the sequence about 2,500 bp in the upstream of transcription starting point (marked as +1) to implement direct sequencing or clone sequencing and compared the sequencing results.

TABLE 2
Primer Sequences and Location for Clone of Upstream Sequence of
KAP8 Gene

| | Name | | | SEQ |
|---|---|---|---|---|
| Amplification | of | | Expected | ID |
| Region | Primer | Primer Sequence (5′-3′) | Size (bp) | NO: |
| -2419~-1876 | KAP8 | CCTTTAGGATTGACTGATTT | 544 | 11 |
| | Ef1 | | | |
| | KAP8 | TTTCACTCTCTCTTTCACAA | 12 | |
| | Er1 | | | |
| -1889~-1025 | KAP8 | AAGAGAGAGTGAAAATCGCT | 865 | 13 |
| | Ff1 | | | |
| | KAP8 | ATGAATGGGAACTTTTACCT | 14 | |
| | Fr1 | | | |
| -1099~-232 | KAP8 | ATTTGTTACATAATCTGGTT | 868 | 15 |
| | Gf1 | | | |
| | KAP8 | CCTGGGTCTTATAAAGTCCT | 16 | |
| | Gr1 | | | |
| -565~155 | KAP8 | AACAACCCATCCTAGTATTC | 720 | 17 |
| | Hf1 | | | |
| | KAP8 | GGAGTAGGTACTACCGTAGC | 18 | |
| | Hr1 | | | |

Common PCR reaction system (20 μL) includes 2.0 μL 10× buffer (including MgSO₄), 1.6 μL dNTP (2.5 mM), each of 2.0 μL upstream and downstream primer (10 μM) respectively, 0.4 μL Taq polymerase (5 U/μL), 9 μL deionized water and 3 μL DNA template.

Optimized PCR reaction procedures are:

Denaturation for 5 min at 95° C.; denaturation for 30 s at 95° C., annealing for 30 s (annealing temperature is 54.5° C. for Efl pair, 47.5° C. for Ffl pair, 52.5° C. for Gfl pair and 54.5° C. for Hfl pair), and extension for 35 s at 72° C. which serve as a cycle, with implementing 30 cycles; then extension for 5min at 72° C.; and then cool down at 4° C.

The PCR products were detected on 1.5% agarose gel and electrophoresis images were photoed by gel imaging system for storage with 4S Red as the nucleic acid dye. The positive PCR products were delivered to Hangzhou QingKe Biotechnology Company for direct sequencing using corresponding PCR primers as the sequencing primers. If there was the base that could not be identified in the sequencing product, cloning sequencing technology should be adopted as the alternative technology.

During the sequencing for PCR products of two primer sets, a fragment insertion-deletion polymorphism of about 7 bases was identified in Chinese Merino fine wool sheep and Suffolk sheep, that is, 868 bp and 720 bp PCR products obtained from these two breeds. Furthermore, the insertion-deletion polymorphism was (CA) n repeat sequence, and the last CA repeat was 458 bp away from the transcription starting point. Detailed analysis found that n, the number of repeat CA was 17 or 18 in Chinese Merino fine wool sheep, while the number of repeat CA was 23 or 24 in Suffolk sheep.

### IMPLEMENTATION EXAMPLE 5

### Analysis of STR Locus in Upstream Region of KAP8 Gene in Multiple sheep Breeds

In order to learn more about the polymorphism of CA repeat sequence found in Example 4 in different sheep breeds, primer set of KAP8 Gfl-KAP8 Grl was selected as the optimal primer by comparing the PCR amplification conditions and the sequencing results. This selected primer set was used to amplify DNA samples and sequence PCR products in 6 flocks, which included Chinese Merino fine wool sheep, Suffolk, Hu Sheep, Chinese Merino fine wool hybrid meat strain, Chinese Merino meat-prolificacy strain crossed with Hu Sheep and goat. Comparison of sequence differences and CA repeat number was carried out in the DNA samples of all individual.

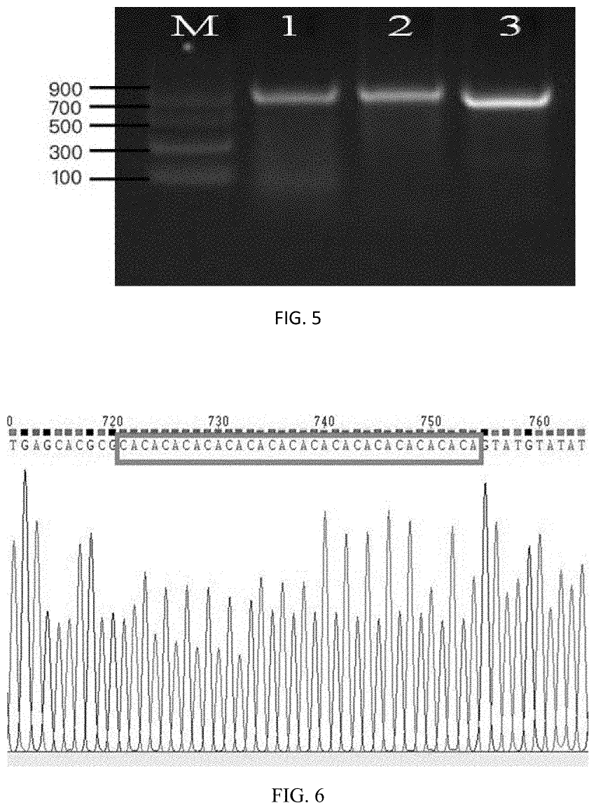

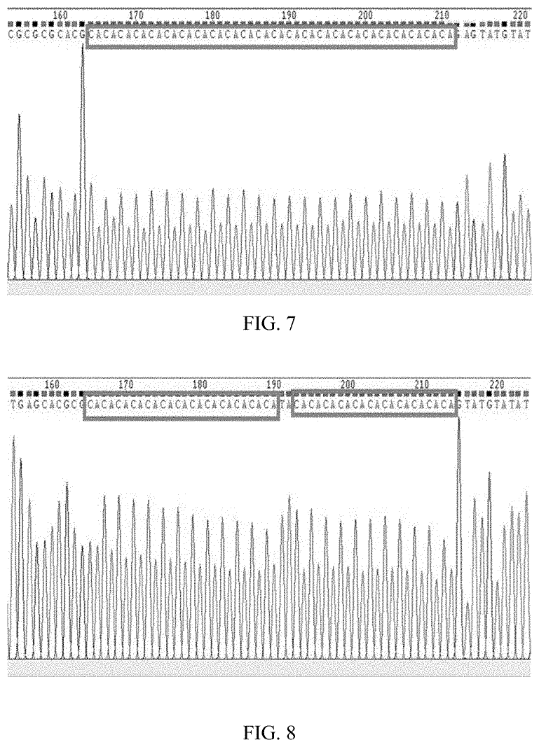

The PCR products obtained from all sheep DNA samples were basically consistent with the expected size 868 bp, while the products obtained from goat DNA were smaller than those obtained from sheep DNA, therefore, the electrophoretic band was located below that of sheep (showed in FIG. 5 for part of the electrophoresis results). The sequencing results showed that the number of repeat CA in Chinese Merino fine wool sheep was 17 (FIG. 6) or 18; the number of repeat CA in Suffolk was 23 or 24 (FIG. 7); the number of repeat CA in Hu Sheep was 24; the number of repeat CA in goat was 5. In view of the change in the length of repeat sequence, the PCR product of goat was less than 12 or 13 CA repeat than that of Suffolk, therefore, the PCR product was smaller, which was consistent with the electrophoresis results. However, the sum of the number of CA repeat sequence in Chinese Merino fine wool hybrid meat strain was 23 or 24, while the CA repeat was discontinuous with dividing two segments by two basic groups, TA or GA, in the number 12+11 or 13+11; the sum of the number of CA repeat sequence in Chinese Merino meat-prolificacy strain was also 23 or 24, and the CA repeat was discontinuous with dividing two segments by two basic groups, TA or GA, in the number 12+11 or 13+11 (FIG. 8).

It can be known from the above implementation examples that when the number of CA repeat sequence is 17 or 18, the sheep to be tested is fine-wool sheep, and when the number of CA repeat sequences is 23 or 24, the sheep to be tested is non-fine-wool sheep; when the number of CA repeat sequence is discontinuous repeat, the sheep to be tested is the hybrid offspring of fine-wool sheep with non-fine-wool sheep. Therefore, the above result is directly related to the breed of fine-wool sheep, and the number of CA repeat can be directly used for the identification and assisted breeding of the breed of fine-wool sheep.

The above detailed examples illustrate that the STR locus found in the invention has high specificity, after multiple analysis and screening for KAP8 gene. And this STR locus is a better choice of molecular marker for the identification of fine-wool sheep breed and assisted fine wool sheep crossbreeding.

What is claimed is:

1. A STR molecular marker in a KAP8 gene of sheep for assisted breeding of fine-wool sheep, comprising a CA base repeat having a repeat number in an upstream region of the KAP8 gene of sheep, wherein the repeat number is different between fine-wool sheep and other sheep breeds.

2. The STR molecular marker described in claim 1, wherein the repeat number of its CA base repeat between 5 and 24.

3. The STR molecular marker described in claim 1, wherein the last CA repeat is 458 bp from a KAP8 transcription starting point.

4. The STR molecular marker described in claim 1, wherein the repeat number of its CA base repeat between 5 and 24, the last CA repeat is 458 bp from the KAP8 transcription starting point, and which is usable as a DNA molecular marker for breeding of fine-wool sheep, identification of fine-wool sheep and non-fine-wool sheep breeds, and identification of hybrid offspring of fine-wool sheep and non-fine-wool sheep.

5. A primer pair for detecting the STR molecular marker of claim 1, comprising a first primer consisting essentially of SEQ ID NO: 1 and a second primer consisting essentially of SEQ ID NO: 2.

6. A method for identifying fine-wool sheep comprising amplifying a STR molecular marker in a KAP8 gene of sheep, the STR marker comprising a (CA)ₙ repeat having a repeat number n in an upstream region of the KAP8 gene of sheep.

7. The method of claim 6, wherein n is between 5 and 24.

8. The method of claim 6, wherein the last CA repeat is 458 bp from a KAP8 transcription starting point.

9. The method of claim 7, wherein when n is 17 or 18, the sheep is a fine-wool breed; when n is 23 or 24, the sheep is a non-fine-wool breed; and when the CA repeat sequence number is in the range 5≤n≤24, and is discontinuous, the sheep is a hybrid offspring of fine-wool breed and non-fine-wool breed.

10. The method of claim 9, wherein when n is 17 or 18, the sheep is a fine-wool breed; when n is 23 or 24, the sheep is a non-fine-wool breed; and when the CA repeat sequence number is in the range 5≤n≤24, and is discontinuous, the sheep is a hybrid offspring of fine-wool breed and non-fine-wool breed.

11. The method described in patent claim 6, comprising the following steps:
1) obtaining genomic DNA from a sheep to be tested;
2) amplifying, by PCR amplification, the DNA obtained in step 1) using primers designed to amplify the STR molecular marker;
3) sequencing the PCR amplification product obtained in step 2) to determine the (CA)ₙ repeat number n; wherein when n is 17 or 18, the sheep is a fine-wool breed; when n is 23 or 24, the sheep is a non-fine-wool breed; and when n is in the range 5≤n≤24, and is discontinuous, the sheep is a hybrid offspring of a fine-wool breed and a non-fine-wool breed.

12. The method of claim 6, comprising using a primer pair, comprising a first primer consisting essentially of the sequence of SEQ ID NO.1 and a second primer consisting essentially of the sequence of SEQ ID NO: 2 to amplify the STR marker.

13. The method of claim 11, further comprising in step 2), using a primer pair, comprising a first primer consisting essentially of the sequence of SEQ ID NO.1 and a second primer consisting essentially of the sequence of SEQ ID NO: 2 to amplify the STR marker.

14. The method of claim 13, wherein the PCR amplification of step 2) is performed in a total volume of 20 μL, including 2.0 μL buffer solution 10× including 20 mM MgCl₂, 0.1 μL˜3 μL specific primer pair SEQ ID NO. 1 and SEQ ID NO. 2 at a concentration of 20 μM, 0.2 μL˜4 μL dNTP mix with the concentration of 2.5 mM, 1.0 μL˜5 μL comprising about 40 ng genomic DNA, 0.2 μL˜1 μL Taq DNA polymerase (5 U/μL) and ddH₂O until to 20 μL; and wherein PCR reaction conditions comprise denaturation for 5 min at 95° C.; denaturation for 30 s at 95° C., annealing for 30 s˜40 s at 47° C.˜55° C. and extension 30 s˜40 s at 72° C., as a cycle, for 28˜35 cycles; followed by 5 min extension at 72° C.; and cool down at 4° C.

15. The method of claim 11, wherein the PCR amplification system described in step 2) is performed in a total volume of 20 μL, including 2.0 μL 10× buffer solution including 20 mM MgCl₂, 0.1 μL specific primer pair SEQ ID NO. 1 and SEQ ID NO. 2 at a concentration of 20 μM, 1.2 μL 2.5 mM dNTP mix, 0.2 μL 5 U/μL Taq DNA polymerase, 2 μL 40 ng DNA template and water; wherein PCR reaction conditions comprise denaturation for 5 min at 95° C.; denaturation for 30 s at 95° C., annealing for 30 s at 52.5° C. and extension 35 s at 72° C., as a cycle, for 30 cycles; followed by 5 min extension at 72° C.; and then cool down at 4° C.
